(19) United States
(12) Patent Application Publication
(10) Pub. No.: US 2005/0276768 A1
(43) Pub. Date: Dec. 15, 2005

(54) Multi-phased personal care composition

(76) Inventors: Karl Shiqing Wei, Mason, OH (US); Cheyne Pohlman Thomas, Independence, KY (US); Andres Ernesto Velarde, Cincinnati, OH (US); Edward Dewey Smith III, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/149,644

(22) Filed: Jun. 10, 2005

Related U.S. Application Data
(60) Provisional application No. 60/579,399, filed on Jun. 14, 2004.

(51) Int. Cl. A61K 7/021

(52) U.S. Cl. 424/63

(57) ABSTRACT

The present invention is a multi-phased personal care composition that contains at least two visually distinct phases. At least one visually distinct phase contains a cleansing phase and at least one visually distinct phase contains a colorant. The colorant is substantially free of Barium and/or Aluminum. The phases are packaged in physical contact with one another

### CROSS REFERENCE TO RELATED APPLICATION(S)

This application claims the benefit of U.S. Provisional Application No. 60,579,399, filed Jun. 14, 2004.

### FIELD OF THE INVENTION

The present invention relates to a multi-phased personal care composition comprising at least two visually distinct phases. At least one visually distinct phase comprises a cleansing phase and at least one visually distinct phase comprises a colorant wherein the colorant is substantially free of Barium and/or Aluminum. The phases are packaged in physical contact with one another.

### BACKGROUND OF THE INVENTION

Personal care compositions are becoming more popular in the United States and around the world. Personal care compositions are well known and widely used. Desirable personal care composition must meet a number of criteria. For example, in order to be acceptable to consumers, a personal care composition must exhibit good cleaning properties, must exhibit good lathering characteristics, must be mild to the skin (not cause drying or irritation) and preferably should provide a conditioning benefit to the skin. Personal care compositions have also been used to alter the color and appearance of skin.

Personal care compositions that attempt to provide skin-conditioning benefits with other similar materials in combination with a lathering surfactant are well known. Although these products provide both conditioning and cleansing benefits, it is often difficult to formulate a product that has cleansing and conditioning compositions physically in contact with one another but at the same time visually distinct and stable.

One attempt at providing conditioning and cleansing benefits from a single personal cleansing product while maintaining stability has been the use of dual-chamber packaging. These packages comprise separate cleansing compositions and conditioning compositions, and allow for the co-dispensing of the two in a single or dual stream. The separate compositions thus remain physically separate and stable during prolonged storage and just prior to application, but then mix during or after dispensing to provide conditioning and cleansing benefits from a physically stable system. Although such dual-chamber delivery systems provide improved conditioning benefits over the use of conventional systems, it is often difficult to achieve consistent and uniform performance because of the uneven dispensing ratio between the cleansing phase and the conditioning phase from these dual-chamber packages.

Accordingly, the need still remains for multi-phased personal care composition that comprises at least two visually distinct phases in physical contact that remain stable for long periods of time while at the same time the two visually distinct phases maintain a pattern and still able to effectively deposit conditioning benefits to the user's skin.

It is therefore an object of the present invention to provide a multi-phased personal care composition comprising at least two visually distinct phases. At least one visually distinct phase comprises a cleansing phase and at least one visually distinct phase comprises a colorant wherein the colorant is substantially free of Barium and/or Aluminum. The phases are packaged in physical contact with one another. The use of a colorant substantially free of Barium and/or Aluminum provides improved stability of the cleansing phase so that the cleansing phase can be combined with a second visually distinct phase that can comprise high levels of benefit components. This allows for the maintaining of the stability of the composition while at the same time the benefit components can be deposited at higher levels.

### SUMMARY OF THE INVENTION

The present invention relates to a multi-phase personal care composition comprising: at least two visually distinct phases; wherein at least one visually distinct phase comprises a cleansing phase; and wherein at least one visually distinct phase comprises a colorant wherein the colorant is substantially free of Barium and/or Aluminum; and wherein said phases are packaged in physical contact with one another.

The present invention further relates to a multi-phase personal care composition comprising: at least two visually distinct phases; wherein at least one visually distinct phase comprises a cleansing phase comprising; a) a surfactant component comprising; i) at least one nonionic surfactant having an HLB from about 3.4 to about 15.0; ii) at least one anionic surfactant; iii) at least one amphoteric surfactant; b) an electrolyte; and wherein at least one visually distinct phase comprises a benefit phase; and wherein at least one visually distinct phase contains a colorant wherein said colorant is substantially free of Barium and/or Aluminum; and wherein said phases are packaged in physical contact with one another.

The present invention is also directed to a method of cleansing, moisturizing and delivering skin benefit agents to the skin by applying to the skin a composition as described above.

### DETAILED DESCRIPTION OF THE INVENTION

The multi-phased personal care composition of the present invention comprises at least two visually distinct phases; wherein at-least one visually distinct phase comprises a cleansing phase; and wherein at least one visually distinct phase comprises a colorant wherein said colorant is substantially free of Barium and/or Aluminum; and wherein said phases are packaged in physical contact with one another.

These and other essential limitations of the compositions and methods of the present invention, as well as many of the optional ingredients suitable for use herein, are described in detail hereinafter.

The term “anhydrous” as used herein, unless otherwise specified, refers to those compositions or materials containing less than about 10%, more preferably less than about 5%, even more preferably less than about 3%, even more preferably zero percent, by weight of water.

The term “ambient conditions” as used herein, refers to surrounding conditions at one (1) atmosphere of pressure, 50% relative humidity, and 25° C.

The term “cosmetically efficacious level” as used herein, is a level conferring a benefit derived from use of the composition.

The term “domain”, as used herein means a volume of material, component, composition or phase comprising a molecular mixture which can be concentrated but not further separated by physical forces such as ultracentrifugation. For example, surfactant lamellar, surfactant micellar, surfactant crystal, oil, wax, water-glycerine mixture, hydrated hydrophilic polymer all constitute domains which can be concentrated and observed by ultracentrifugation, but which cannot be further separated into distinct molecular components by the same forces.

The term “hydrophobically modified interference pigment” or “HMIP”, as used herein, means a portion of the interference pigment surface has been coated, including both physical and chemical bonding of molecules, with a hydrophobic material.

The term “interference pigment”, as used herein, means a pigment with pearl gloss prepared by coating the surface of a particle substrate material (generally platelet in shape) with a thin film. The thin film is a transparent or semitransparent material having a high refractive index. The higher refractive index material shows a pearl gloss resulting from mutual interfering action between reflection and incident light from the platelet substrate/coating layer interface and-reflection of incident light from the surface of the coating layer.

By the term “multi-phased” or “multi-phase” as used herein, is meant that the at least two phases herein occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phased” personal care compositions comprising at least two phases are present within the container as a visually distinct pattern. The pattern results from the blending of the phases without mixing or homogenization of either one into the other so that neither phase is continuous. The patterns include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, geometric, marbled, and combinations thereof.

In a preferred embodiment the striped pattern may be relatively uniform and even across the dimension of the package. Alternatively, the striped pattern may be uneven, i.e. wavy, or may be non-uniform in dimension. The striped pattern does not need to necessarily extend across the entire dimension of the package. The phases may be various different colors, or include particles, glitter or pearlescence.

The term “multi-phased personal care composition” as used herein, refers to compositions intended for topical application to the skin or hair.

The term “opaque” structured domain as used herein refers to a surfactant domain with ordered structures (e.g., lamellar structure, vesicle structure, cubic structure, etc.) and it is visually opaque to a naked eye in a 10 mm inner diameter plastic centrifuge tube after the Ultracentrifugation Method described herein.

The term “phases” as used herein, refers to a region of a composition having one average composition, as distinct from another region having a different average composition, wherein the regions are visible to the naked eye. This would not preclude the distinct regions from comprising two similar phases where one phase could comprise pigments, dyes, particles, and various optional ingredients, hence a region of a different average composition. Preferably at least one phase comprises a colorant.

The term “Shear Index” or “n” as used herein is a measure of viscosity and is used in combination with Consistency value, to define viscosity for materials whose viscosity is a function of shear rate. The measurements are made at 25° C. and the units of Shear Index are dimensionless and Consistency values are Pascal-seconds (Pa-s).

The term “stable” as used herein, unless otherwise specified, refers to compositions that maintain at least two “separate” phases when sitting in physical contact at ambient conditions for a period of at least about 180 days wherein the distribution of the two phases in different locations in the package does not visibly change over time. By “separate” is meant that the well-distributed nature of the visually distinct phases is compromised, such that larger regions of at least one phase collect until the balanced dispensed ratio of the two or more compositions relative to each other is compromised.

The phrase “substantially free of” as used herein, means that the composition comprises less than about 3%, preferably less than about 1%, more preferably less than about 0.5%, even more preferably less than about 0.25%, and most preferably less than about 0.1%, by weight of the composition, of the stated ingredient.

The phrase “UV stable” as used herein, means stable to UV light exposure. For example, exposure of a composition in a transparent or translucent package to UV light in a Fadometer which utilizes a water cooled xenon lamp to simulate sunlight exposure does not significantly fade the color of the product as determined by a side by side comparison of before and after exposure to 6 hours of light (simulating 30 days sunlight), which can also be determined by having a low delta Ecmc of the colored phase (e.g., delta E may be less that 5, preferably less than 4, more preferably less than 3, still more preferably less than 2 for a 0.012% colorant slurry in a 60% petrolatum, 39.88% Hydrobrite 1000 mineral oil mixture).

All percentages, parts and ratios as used herein are by weight of the total composition, unless otherwise specified. All such weights as they pertain to listed ingredients are based on the active level and, therefore do not include solvents or by-products that may be included in commercially available materials, unless otherwise specified.

The multi-phased personal care composition compositions and methods of the present invention can comprise, consist of, or consist essentially of, the essential elements and limitations of the invention described herein, as well as any additional or optional ingredients, components, or limitations described herein or otherwise useful in personal care compositions intended for topical application to the hair or skin.

Product Form

The multi-phased personal care composition of the present invention is typically in the form of a liquid. The term “liquid” as used herein means that the composition is generally flowable to some degree. “Liquids”, therefore, can include liquid, semi-liquid, cream, lotion or gel compositions intended for topical application to skin. The compositions typically exhibit a viscosity of from about 1,500 cps to about 1,000,000 cps, as measured by the Viscosity Method as described in copending application Ser. No. 60/542,710 filed on Feb. 6, 2004. These compositions contain at least two phases, which are described in greater detail hereinafter.

When evaluating a multi-phased personal care composition, by the methods described herein, preferably each individual phase is evaluated prior to combining, unless otherwise indicated in the individual methodology. However, if the phases are combined, each phase can be separated by centrifugation, ultracentrifugation, pipetting, filtering, washing dilution, concentration, or combination thereof, and then the separate components or phases can be evaluated. Preferably, the separation means is chosen so that the resulting separated components being evaluated is not destroyed, but is representative of the component as it exists in the multi-phased personal care composition. All of the product forms contemplated for purposes of defining the compositions and methods of the present invention are rinse-off formulations, by which is meant the product is applied topically to the skin or hair and then subsequently (i.e., within minutes) the skin or hair is rinsed with water, or otherwise wiped off using a substrate or other suitable removal means with deposition of a portion of the composition.

In a preferred embodiment of the present invention the multi-phased personal care composition, the composition has at least two visually distinct phases wherein at least one phase is visually distinct from a second phase. The visually distinct phases are packaged in physical contact with one another and are stable.

Phases

The multi-phase personal care compositions of the present invention comprise at least two phases, wherein the composition can have a first phase a second phase and so on. The ratio of a first phase to a second phase is about 1:99 to about 99:1, preferably 90:10 to about 10:90, more preferably about 80:20 to about 20:80, even more preferably about 70:30 to about 30:70, still even more preferably about 60:40 to about 40:60, even still even more preferably about 50:50. Each phase could be one or more of the following nonlimiting examples including: a cleansing phase, a benefit phase, which is described in greater detail hereinafter.

Colorants

The multi-phased personal care composition comprises a colorant in at least one phase of the visually distinct phases. The colorants for use in the present invention are stable to UV light exposure. Preferably, the colorant is substantially free of Barium and/or Aluminum. It is believed that the colorant may play an important role in cleansing phase stability by partitioning into the surfactant component which comprises a structured domain. It is also believed that colorants comprising Barium and/or Aluminum aid in degrading this structured domain. The Barium and/or Aluminum interact with the surfactants located in the cleansing phase by interacting with the anionic groups for example sulfates and thereby destabilizing the multi-phased personal care composition.

Using the Ultracentrifugation Method, which determines the percent of an structured domain that is present in a multi-phased personal care composition, described hereafter, one can demonstrate how much of the structured domain has been degraded when a colorant with Barium and/or Aluminum, is present in the multi-phased personal care composition versus the multi-phased personal care composition with a colorant that is substantially free of Barium and/or Aluminum.

Therefore, the multi-phased personal care composition of the present invention preferably comprises colorants that are substantially free of Barium and/or Aluminum.

The multi-phased personal care composition comprises from about 0.00001% to about 10%, by weight of the composition, of said colorant. Preferably multi-phased personal care composition comprises from about 0.0001% to about 1%, more preferably from about 0.001% to about 0.1%, even more preferably from about 0.005% to about 0.05%, by weight of the composition, of said colorant.

The colorants for use in the multi-phase personal care compositions are selected from the group consisting of organic pigments, inorganic pigments, interference pigments, lakes, natural colorants, pearlescent agents, dyes, carmines, and mixtures thereof.

Non-limiting examples of colorants include: D&C Red 30 Talc Lake, D&C Red 7 Calcium Lake, D&C Red 34 Calcium Lake, Mica/Titanium Dioxide/Carmine Pigments (Clorisonne Red from Engelhard, Duocrome RB from Engelhard, Magenta from Rona, Dichrona RB from Rona), Red 30 Low Iron, D&C Red Lake Blend of Lake 27 & Lake 30, FD&C Yellow 5 Lake, Kowet Titanium Dioxide, Yellow Iron Oxide, D&C Red 30 Lake, D&C Red 28 Lake, Cos Red Oxide BC, Cos Iron Oxide Red BC, Cos Iron oxide Black BC, Cos Iron Oxide Yellow, Cos Iron Oxide Brown, Cos Iron Oxide Yellow BC, Euroxide Red Unsteril, Euroxide Black Unsteril, Euroxide Yellow Steril, Euroxide Black Steril, Euroxide Red, Euroxide Black, Hydrophobic Euroxide Black, Hydrophobic Euroxide Yellow, Hydrophobic Euroxide Red, D&C Yellow 6 Lake, D&C Yellow 5 Zr Lake, and mixtures of these colorants.

Cleansing Phase

The multi-phase personal care composition of the present invention comprises a cleansing phase. The cleansing phase comprises a surfactant component or mixtures of surfactants. The multi-phased personal care composition comprises from about 1% to about 95%, by weight of the composition, of said cleansing phase.

Surfactant Component

The surfactant component comprises a surfactant or a mixture of surfactants. The surfactant component comprises surfactants suitable for application to the skin or hair. Suitable surfactants for use herein include any known or otherwise effective cleansing surfactant suitable for application to the skin, and which is otherwise compatible with the other essential ingredients in the multi-phased personal care composition including water. These surfactants include anionic, nonionic, cationic, zwitterionic or amphoteric surfactants, soap or combinations thereof.

The multi-phased personal care composition preferably comprises a surfactant component at concentrations ranging from about 10% to about 99%, more preferably from about 25% to about 90%, even more preferably from about 35% to about 88%, still more preferably from about 40% to about 85%, and still even more preferably from about 45% to about 85%, by weight of the multi-phased personal care composition. The preferred pH range of the multi-phased personal care composition is from about 5 to about 8, more preferably about 6. The surfactant component in the present invention exhibits Non-Newtonian shear thinning behavior.

The surfactant component comprises a structured domain comprising a structured surfactant system. The structured domain enables the incorporation of high levels of benefit components in a separate phase that are not emulsified in the composition but suspended. In a preferred embodiment the structured domain is an opaque structured domain. The opaque structured domain is preferably a continuous lamellar phase. The lamellar phase produces a lamellar gel network that is a type of colloidal system. The lamellar phase provides resistance to shear, adequate yield to suspend particles and droplets and at the same time provides long term stability, since they are thermodynamically stable. The lamellar phase yields a higher viscosity without the need for viscosity modifiers. The multi-phased personal care composition comprising the surfactant component has a Structured Domain Volume Ratio of at least about 45%, preferably at least about 50%, more preferably at least about 55%, even more preferably at least about 60%, still more preferably at least about 65%, still even more preferably at least about 70%, and still even still more preferably at least about 80% as measured by the Ultracentrifugation Method described hereafter.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678.

Anionic surfactants suitable for use in the cleansing phase include alkyl and alkyl ether sulfates. These materials have the respective formula ROSO₃M and RO(C₂H₄O)_{X}SO₃M, wherein R is alkyl or alkenyl of from about 8 to about 24 carbon atoms, x is 1 to 10, and M is a water-soluble cation such as ammonium, sodium, potassium and triethanolamine. The alkyl ether sulfates are typically made as condensation products of ethylene oxide and monohydric alcohols having from about 8 to about 24 carbon atoms. Preferably, R has from about 10 to about 18 carbon atoms in both the alkyl and alkyl ether sulfates. The alcohols can be derived from fats, e.g., coconut oil or tallow, or can be synthetic. Lauryl alcohol and straight chain alcohols derived from coconut oil are preferred herein. Such alcohols are reacted with about 1 to about 10, preferably from about 3 to about 5, and more preferably with about 3, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the cleansing phase are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [R¹-SO₃-M], wherein R¹ is chosen from the group consisting of a straight or branched chain, saturated aliphatic hydrocarbon radical having from about 8 to about 24, preferably about 10 to about 18, carbon atoms; and M is a cation. Suitable examples are the salts of an organic sulfuric acid reaction product of a hydrocarbon of the methane series, including iso-, neo-, ineso-, and n-paraffins, having about 8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C₁₀-₁₈ n-paraffins.

Preferred anionic surfactants for use in the cleansing phase include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium tridecyl benzene sulfonate, sodium dodecyl benzene sulfonate, and combinations thereof.

Anionic surfactants with branched alkyl chains such as sodium trideceth sulfate, for example, are preferred in some embodiments. Mixtures of anionic surfactants may be used in some embodiments.

Additional surfactant from the classes of amphoteric, zwitterionic surfactant, cationic surfactant, and/or nonionic surfactant may be incorporated in the cleansing phase compositions.

Amphoteric surfactants suitable for use in the cleansing phase include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylarninopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378.

Zwitterionic surfactants suitable for use in the cleansing phase include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula: 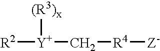
wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use in the cleansing phase include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

Amphoacetates and diamphoacetates may also be used. 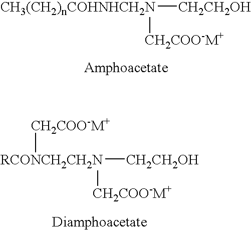
Amphoacetates and diamphoacetates conform to the formulas (above) where R is an aliphatic group of 8 to 18 carbon atoms. M is a cation such as sodium, potassium, ammonium, or substituted ammonium. Sodium lauroamphoacetate, sodium cocoamphoactetate, disodium lauroamphoacetate, and disodium cocodiamphoacetate are preferred in some embodiments.

Cationic surfactants can also be used in the cleansing phase, but are generally less preferred, and preferably represent less than about 5% by weight of the compositions.

Suitable nonionic surfactants for use in the aqueous cleansing phase include condensation products of alkylene oxide groups (hydrophilic in nature) with an organic hydrophobic compound, which may be aliphatic or alkyl aromatic in nature.

In an alternate embodiment of the present invention the cleansing phase comprises a surfactant component comprising a mixture of at least one nonionic surfactant, at least one anionic surfactant and at least one amphoteric surfactant, and an electrolyte.

Non-Ionic Surfactants

In an alternate embodiment of the present invention the multi-phased personal care composition can comprises at least one nonionic surfactant. Preferably the nonionic surfactant has an HLB from about 1.0 to about 15.0, preferably from about 3.4 to about 15.0, more preferably from about 3.4 to about 9.5, even more preferably from about 3.4 to about 5.0. The multi-phased personal care composition preferably comprises a nonionic surfactant at concentrations ranging from about 0.01% to about 50%, more preferably from about 0.10% to about 10%, and even more preferably from about 0.5% to about 5.0%, by weight of the surfactant component.

Nonionic surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Non-limiting examples of preferred nonionic surfactants for use herein are those selected form the group consisting of C₈-C₁₄ glucose amides, C₈-C₁₄ alkyl polyglucosides, sucrose cocoate, sucrose laurate, and mixtures thereof. In a preferred embodiment the nonionic surfactant is selected from the group consisting of glyceryl monohydroxystearate, Steareth-2, hydroxy stearic acid, propylene glycol stearate, PEG-2 stearate, sorbitan monostearate, glyceryl stearate, laureth-2 and mixtures thereof. In a preferred embodiment the nonionic surfactant is Steareth-2.

Nonionic surfactants also useful herein include, lauramine oxide, cocoamine oxide.

Anionic Surfactants

In the alternate embodiment of the present invention the multi-phased personal care composition can comprises at least one anionic surfactant. Nonlimiting examples of suitable anionic surfactant were discussed previously.

Amphoteric Surfactants

In the alternate embodiment of the present invention the multi-phased personal care composition can comprises at least one amphoteric surfactant. Nonlimiting examples of suitable amphoteric surfactant were discussed previously.

Electrolyte

The electrolyte, if used, can be added per se to the multi-phased personal care composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium or ammonium chloride or sodium or ammonium sulfate. A preferred electrolyte is sodium chloride. The electrolyte is preferably added to the surfactant component of the composition.

The electrolyte, when present, should be present in an amount, which facilitates formation of the stable composition (Non-Newtonian shear thinning behavior). Generally, this amount is from about 0.1% by weight to about 15% by weight, preferably from about 1% to about 6% by weight of the multi-phased personal care, but may be varied if required.

In another alternative embodiment of the present invention, the surfactant for use in the cleansing phase can be mixtures of surfactants. Suitable surfactant mixtures can comprise water, at least one anionic surfactant as described previously, an electrolyte as described previously, and at least one alkanolamide. The alkanolamide if present has the general structure of: 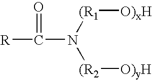
wherein R is C₈ to C₂₄, or preferably in some embodiments C₈ to C₂₂ or in other embodiments C₁₈ to C₁₈, saturated or unsaturated, straight chain or branched, aliphatic group; R₁ and R₂ are the same or different C₂-C₄ straight chain or branched aliphatic group; x is from 0 to 10; y is from 1 to 10; and wherein the sum of x and y is less than or equal to 10.

The amount of alkanolamide in the composition is typically about 0.1% to about 10%, by weight of the lathering cleansing phase, and in some embodiments is preferably from about 2% to about 5%, by weight of the lathering cleansing phase. Suitable alkanolamides include Cocamide MEA (Coco monethanolamide) and Cocamide MIPA (Coco monoisopropranolamide).

Lamellar Builder

The cleansing phase of the present compositions optionally, but preferably, further comprise about 0.1% to 10% by wt. of a lamellar builder which functions in the compositions to form a thermodynamic domain, preferably a lamellar(structured) domain. It is believed the lamellar domain enhances the interfacial stability between the phases of the present compositions.

Suitable lamellar builders include a fatty acid or ester derivatives thereof, a fatty alcohol, trihydroxystearin (available from Rheox, Inc. under the trade name THIXCIN® R), or polymethacrylamidopropyltrimonium chloride (available from Rhodia under the trade name POLYCARE® 133). Preferably, the lamellar builder is selected from lauric acid or trihydroxystearin.

Benefit Phase

The multi-phase personal care compositions of the present invention can comprise a benefit phase. The benefit phase in the present invention is preferably anhydrous. The benefit phase comprises hydrophobic compositions comprising hydrophobic components. The benefit phase comprises from about 20% to about 100%, preferably at least about 35%, most preferably at least about 50% of a hydrophobic component. The hydrophobic compositions are preferably selected among those having defined rheological properties as described in copending application Ser. No. 60/542,710 filed on Feb. 6, 200, including selected Consistency value (k) and Shear Index (n). These preferred rheological properties are especially useful in providing the multi-phased personal care composition compositions with improved deposition of hydrophobic components on the skin.

Nonlimiting examples of hydrophobic components suitable for use herein can include a variety of hydrocarbons, oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, polyglycerin fatty acid esters, lanolin and its derivatives, wax esters, beeswax derivatives, sterols and phospholipids, vitamins and pro-vitamins and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, and combinations thereof.

Non-limiting examples of silicone oils suitable for use as hydrophobic components herein include dimethicone copolyol, dimethylpolysiloxane, diethylpolysiloxane, mixed C1-C30 alkyl polysiloxanes, phenyl dimethicone, dimethiconol, and combinations thereof. Preferred are non-volatile silicones selected from dimethicone, dimethiconol, mixed C1-C30 alkyl polysiloxane, and combinations thereof. Nonlimiting examples of silicone oils useful herein are described in U.S. Pat. No. 5,011,681 (Ciotti et al.).

Non-limiting examples of diglycerides and triglycerides suitable for use as hydrophobic components herein include castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof.

Non-limiting examples of acetoglyceride esters suitable for use as hydrophobic components herein include acetylated monoglycerides.

Non-limiting examples of alkyl esters suitable for use as hydrophobic components herein include isopropyl esters of fatty acids and long chain esters of long chain (i.e. C₁₀-C₂₄) fatty acids, e.g. cetyl ricinoleate, non-limiting examples of which include isopropyl palmitate, isopropyl myristate, cetyl riconoleate and stearyl riconoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as hydrophobic components herein include oleyl myristate, oleyl stearate, oleyl oleate, and combinations thereof.

Non-limiting examples of polyglycerin fatty acid esters suitable for use as hydrophobic components herein include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as hydrophobic components herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Still other suitable hydrophobic components include milk triglycerides (e.g., hydroxylated milk glyceride) and polyol fatty acid polyesters.

Still other suitable hydrophobic components include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

In a preferred embodiment the multi-phased personal care composition can comprise a multi-phase composition having a benefit phase; wherein the benefit phase preferably can comprise one or more hydrophobic components, wherein at least 20% by weight of the hydrophobic components are selected from petrolatum, mineral oil, sunflower seed oil, micro-crystalline waxes, paraffins, ozokerite, polyethylene, polybutene, polydecene and perhydrosqualene dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, castor oil, soy bean oil, maleated soy bean oil, safflower oil, cotton seed oil, corn oil, walnut oil, peanut oil, olive oil, cod liver oil, almond oil, avocado oil, palm oil and sesame oil, and combinations thereof. More preferably, at least about 50% by weight of the hydrophobic components are selected from the groups of petrolatum, mineral oil, paraffins, polyethylene, polybutene, polydecene, dimethicones, alkyl siloxanes, cyclomethicones, lanolin, lanolin oil, lanolin wax. The remainder of the hydrophobic components is preferably selected from: isopropyl palmitate, cetyl riconoleate, octyl isononanoate, octyl palmitate, isocetyl stearate, hydroxylated milk glyceride and combinations thereof.

Particle

The multi-phased personal care composition can comprise a particle. Water insoluble solid particles of various shapes and densities are useful. In a preferred embodiment, the particle tends to have a spherical, an oval, an irregular, or any other shape in which the ratio of the largest dimension to the smallest dimension (defined as the Aspect Ratio) is less than about 10. More preferably, the Aspect Ratio of the particle is less than about 8, still more preferably the Aspect Ratio of the particle is less than about 5.

The multi-phased personal care composition of the present invention comprises the particle at a cosmetically efficacious level. Preferably, the particles are present from at least about 0.1% by weight of the composition, more preferably at least about 0.2% by weight of composition, even more preferably at least about 0.5%, still more preferably at least about 1%, and even still more preferably at least 2% by weight of composition. In the multi-phased personal care composition of the present invention, preferably the particles comprises no more than about 50% by weight of composition, more preferably no more than about 30%, still more preferably no more than about 20%, and even more preferably no more than about 10% by weight of composition.

Preferably, the particle will also have physical properties which are not significantly affected by typical processing of the composition. Preferably, a particle having a melting point greater than about 70° C. is used, more preferably having a melting point greater than about 80° C., and even more preferably having a melting point of greater than about 95° C. is used. As used herein, melting point would refer to the temperature at which the particle transitions to a liquid or fluid state or undergoes significant deformation or physical property changes. In addition, many of the particles of present invention are cross-linked or have a cross-linked surface membrane. These particles do not exhibit a distinct melting point. Cross-linked particles are also useful as long as they are stable under the processing and storage conditions used in the making of compositions.

The particles that can be present in the present invention can be natural, synthetic, or semi-synthetic. In addition, hybrid particles can also be present. Synthetic particles can made of either cross-linked or non cross-linked polymers. The particles of the present invention can have surface charges or their surface can be modified with organic or inorganic materials such as surfactants, polymers, and inorganic materials. Particle complexes can be present.

Non limiting examples of natural particles include various precipitated silica particles in hydrophilic and hydrophobic forms available from Degussa-Huls under the trade name Sipernet. Precipitated™, hydrophobic, synthetic amorphous silica, available from Degussa under the trade name Sipernet D11™ is a preferred particle. Snowtex colloidal silica particles are available from Nissan Chemical America Corporation.

Nonlimiting examples of synthetic particles include nylon, silicone resins, poly(meth)acrylates, polyethylene, polyester, polypropylene, polystyrene, polyurethane, polyamide, epoxy resins, urea resins, and acrylic powders. Non limiting examples of useful particles are Microease 110S, 114S, 116 (micronized synthetic waxes), Micropoly 210, 250S (micronized polyethylene), Microslip (micronized polytetrafluoroethylene), and Microsilk (combination of polyethylene and polytetrafluoroethylene), all of which are available from Micro Powder, Inc. Additional examples include Luna (smooth silica particles) particles available from Phenomenex, MP-2200 (polymethylmethacrylate), EA-209 (ethylene/acrylate copolymer), SP-501(nylon-12), ES-830 (polymethly methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful.

Non limiting examples of hybrid particles include Ganzpearl GSC-30SR (Sericite & crosslinked polystyrene hybrid powder), and SM-1000, SM-200 (mica and silica hybrid powder available from Presperse).

Exfoliant Particle

The exfoliant particle is selected from the group consisting of polyethylene, microcrystalline wax, jojoba esters, amorphous silica, talc, tracalcium orthophosphate, or blends thereof, and the like. The exfoliant particle has a particle size dimension along the major axis of the particle of from about 100 microns to about 600 microns, preferably from about 100 microns to about 300 microns. The exfoliant particle has a hardness of less than about 4 Mohs, preferably less than about 3 Mohs. The hardness as so measured is a criterion of the resistance of a particular material to crushing. It is known as being a fairly good indication of the abrasive character of a particulate ingredient. Examples of materials arranged in increasing order of hardness according to the Moh scale are as follows: h(hardness)-1:talc; h-2: gypsum, rock salt, crystalline salt in general, barytes, chalk, brimstone; h-4: fluorite, soft phosphate, magnesite, limestone; h-5: apatite, hard phosphate, hard limestone, chromite, bauxite; h-6: feldspar, ilmenite, hornblendes; h-7: quartz, granite; h-8: topaz; h-9: corrundum, emery; and h-10: diamond.

Preferably, the exfoliant particle has a color distinct from the cleansing phase. The exfoliant particle is preferably present at a level of less than about 10%, preferably less than about 5%, by wt of the composition.

Shiny Particles

The multi-phased personal care composition can comprise a shiny particle. In a preferred embodiment, the shiny particle is in at least one phase of the multi-phase personal care composition. Nonlimiting examples of shiny particles include the following: interference pigment, multi-layered pigment, metallic particle, solid and liquid crystals, or combinations thereof.

An interference pigment is a pigment with pearl gloss prepared by coating the surface of a particle substrate material with a thin film. The particle substrate material is generally platelet in shape. The thin film is a transparent or semitransparent material having a high refractive index. The high refractive index material shows a pearl gloss resulting from mutual interfering action between reflection and incident light from the platelet substrate/coating layer interface and reflection of incident light from the surface of the coating layer. The interference pigments of the multi-phased personal care compositions preferably comprises no more than about 20 weight percent of the composition, more preferably no more than about 10 weight percent, even more preferably no more than about 7 weight percent, and still more preferably no more than about 5 weight percent of the multi-phased personal care composition. The interference pigment of the multi-phased personal care composition preferably comprises at least about 0.1 weight percent of the multi-phased personal care composition, more preferably at least about 0.2 weight percent, even more preferably at least about 0.5 weight percent, and still more preferably at least about 1 weight percent by weight of the multi-phased personal composition.

The interference pigment can comprise a multilayer structure. The centre of the particulates is a flat substrate with a refractive index (RI) normally below 1.8. A wide variety of particle substrates are useful herein. Nonlimiting examples are natural mica, synthetic mica, graphite, talc, kaolin, alumina flake, bismuth oxychloride, silica flake, glass flake, ceramics, titanium dioxide, CaSO₄, CaCO₃, BaSO₄, borosilicate and mixtures thereof, preferably mica, silica and alumina flakes.

A layer of thin film or a multiple layer of thin films are coated on the surface of a substrate described above. The thin films are made of highly refractive materials. The refractive index of these materials is normally above 1.8.

A wide variety of thin films are useful herein. Nonlimiting examples are TiO₂, Fe₂O₃, SnO₂, Cr₂O₃, ZnO, ZnS, ZnO, SnO, ZrO₂, CaF₂, BiOCl, and mixtures thereof or in the form of separate layers, preferably TiO₂, Fe₂O₃, Cr₂O₃ SnO₂. For the multiple layer structures, the thin films can be consisted of all high refractive index materials or alternation of thin films with high and low RI materials with the high RI film as the top layer.

The interference color is a function of the thickness of thin film, the thickness for a specific color may be different for different materials. For TiO₂, a layer of 40 nm to 60 nm or a whole number multiple thereof gives silver color, 60 nm to 80 nm yellow color, 80 nm to 100 nm red color, 100 nm to 130 nm blue color, 130 nm to 160 nm green color. In addition to the interference color, other transparent absorption pigments can be precipitated on top of or simultaneously with the TiO₂ layer. Common materials are red or black iron oxide, ferric ferrocyanide, chromium oxide or carmine. It was found that the color of the interference pigment in addition to its brightness had a significant influence on human perception of skin tone. In general, preferred colors are silver, gold, red, green and mixtures thereof.

Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®.

In an embodiment of the present invention the interference pigment surface is either hydrophobic or has been hydrophobically modified. The hydrophobically modified interference pigment or HMIP allows for the entrapment of the HMIP within the phases and greater deposition of the HMIP. Preferably the ratio of HMIP to a phase is 1:1 to about 1:70, more preferably 1:2 to about 1:50, still more preferably 1:3 to about 1:40 and most preferably 1:7 to about 1:35.

In an embodiment of the present invention the HMIP's are preferably entrapped within the hydrophobic composition. This necessitates that the hydrophobic composition particle size is generally larger than the HMIP. In a preferred embodiment of the invention, the hydrophobic composition particles contain only a small number of HMIPs per hydrophobic composition particles. Preferably this is less than 20, more preferably less than 10, most preferably less than 5. These parameters, the relative size of the benefit droplets to the HMIP and the approximate number of HMIP particles per hydrophobic composition particles, can be determined by using visual inspection with light microscopy.

The HMIP of the present invention preferably has a hydrophobic coating comprising at least about 0.1 weight percent of the total particle weight, more preferably at least about 0.5 weight percent, even more preferably at least about 1 weight percent. Nonlimiting examples of the hydrophobic surface treatment useful herein include silicones, acrylate silicone copolymers, acrylate polymers, alkyl silane, isopropyl titanium triisostearate, sodium stearate, magnesium myristate, perfluoroalcohol phosphate, perfluoropolymethyl isopropyl ether, lecithin, carnauba wax, polyethylene, chitosan, lauroyl lysine, plant lipid extracts and mixtures thereof, preferably, silicones, silanes and stearates. Surface treatment houses include US Cosmetics, KOBO Products Inc., and Cardre Inc.

Optional Ingredients

A variety of suitable optional ingredients can be employed in the multi-phase personal care composition. Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. These optional materials can be used in any aspect of the compositions of the present invention, including each phase as described herein.

Non-limiting optional ingredients include humectants and solutes. A variety of humectants and solutes can be employed and can be present at a level of from about 0.1% to about 50%, preferably from about 0.5% to about 35%, and more preferably from about 2% to about 20%, by weight of the personal care composition. A preferred humectant is glycerin.

A preferred water soluble, organic material is selected from the group consisting of a polyol of the structure:
R1-O(CH₂-CR2HO)ₙH
where R1=H, C1-C4 alkyl; R2=H, CH₃ and n=1-200; C2-C10 alkane diols; guanidine; glycolic acid and glycolate salts (e.g. ammonium and quaternary alkyl ammonium); lactic acid and lactate salts (e.g. ammonium and quaternary alkyl ammonium); polyhydroxy alcohols such as sorbitol, glycerol, hexanetriol, propylene glycol, hexylene glycol and the like; polyethylene glycol; sugars and starches; sugar and starch derivatives (e.g. alkoxylated glucose); panthenol (including D-, L-, and the D,L-forms); pyrrolidone carboxylic acid; hyaluronic acid; lactamide monoethanolamine; acetamide monoethanolamine; urea; and ethanol amines of the general structure (HOCH₂CH₂)ₓNH_{y} where x=1-3; y=0-2, and x+y=3, and mixtures thereof. The most preferred polyols are selected from the group consisting of glycerine, polyoxypropylene(1) glycerol and polyoxypropylene(3) glycerol, sorbitol, butylene glycol, propylene glycol, sucrose, urea and triethanol amine.

Nonionic polyethylene/polypropylene glycol polymers are preferably used as skin conditioning agents. Polymers useful herein that are especially preferred are PEG-2M wherein x equals 2 and n has an average value of about 2,000 (PEG 2-M is also known as Polyox WSR® N-10 from Union Carbide and as PEG-2,000); PEG-5M wherein x equals 2 and n has an average value of about 5,000 (PEG 5-M is also known as Polyox WSR® 35 and Polyox WSR® N-80, both from Union Carbide and as PEG-5,000 and Polyethylene Glycol 200,000); PEG-7M wherein x equals 2 and n has an average value of about 7,000 (PEG 7-M is also known as Polyox WSR® (N-750 from Union Carbide); PEG-9M wherein x equals 2 and n has an average value of about 9,000 (PEG 9-M is also known as Polyox WSR® N-3333 from Union Carbide); PEG-14 M wherein x equals 2 and n has an average value of about 14,000 (PEG 14-M is also known as Polyox WSR-205 and Polyox WSR® N-3000 both from Union Carbide); and PEG-90M wherein x equals 2 and n has an average value of about 90,000. (PEG-90M is also known as Polyox WSR®-301 from Union Carbide.)

Other non limiting examples of these optional ingredients include vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

Ultracentrifugation Method:

The Ultracentrifugation Method is used to determine the percent of a structured domain or an opaque structured domain that is present in a multi-phased personal care composition that comprises a surfactant component. The method involves the separation of the composition through ultracentrifugation into separate but distinguishable layers. The multi-phased personal care composition of the present invention can have multiple distinguishable layers, for example a non-structured surfactant layer, a structured surfactant layer, and a benefit layer.

First, dispense about 4 grams of multi-phased personal care composition into Beckman Centrifuge Tube (11×60 mm). Next, place the centrifuge tubes in an Ultracentrifuge (Beckman Model L8-M or equivalent) and set ultracentrifuge to the following conditions: 50,000 rpm, 18 hours, and 25 C.

After ultracentrifuging for 18 hours, determine the relative phase volume by measuring the height of each layer using an Electronic Digital Caliper (within 0.01 mm). First, the total height is measured as Hₐ which includes all materials in the ultracentrifuge tube. Second, the height of the benefit layer is measured as H_{b}. If there is more than one benefit layer, their heights are combined. Third, the structured surfactant layer is measured as H_{c}. The benefit layer is determined by its low moisture content (less than 10% water as measured by Karl Fischer Titration). It generally presents at the top of the centrifuge tube. The total surfactant layer height (Hₛ) can be calculated by this equation:
Hₛ=Hₐ−H_{b}

The structured surfactant layer components may comprise several layers or a single layer. Upon ultracentrifugation, there is generally an isotropic layer at the bottom or next to the bottom of the ultracentrifuge tube. This clear isotropic layer typically represents the non-structured micellar surfactant layer. The layers above the isotropic phase generally comprise higher surfactant concentration with higher ordered structures (such as liquid crystals). These structured layers are sometimes opaque to naked eyes or translucent. There is generally a distinct phase boundary between the structured layer and the non-structured isotropic layer. The physical nature of the structured surfactant layers can be determined through microscopy under polarized light. The structured surfactant layers typically exhibit distinctive texture under polarized light. Another method for characterizing the structured surfactant layer is to use X-ray diffraction technique. Structured surfactant layer display multiple lines that are often associated primarily with the long spacings of the liquid crystal structure.

Finally, the structured domain volume ratio is calculated based on the following equation:
Structured Domain Volume Ratio=H_{c}/Hₛ*100%

If there is no benefit phase present, use the total height as the surfactant layer height, Hₛ=Hₐ.

The ultracentrifugation method can be used to determine cleansing phase stability during rapid aging at 120 F. The Structured Domain Volume Ratios are determined before and after the rapid aging study. Multiphase compositions in the present invention maintain good phase stability with less than 10% degradation of the structured surfactant layers after 5 days at 120 F. Comparative examples are not stable after the rapid aging study. Some comparative examples show a distinctive phase shift (i.e., formation of a new phase on top of the structured surfactant domain).

Method Of Use

The multi-phase personal care compositions of the present invention are preferably applied topically to the desired area of the skin or hair in an amount sufficient to provide effective delivery of the skin cleansing agent, hydrophobic material, and particles to the applied surface. The compositions can be applied directly to the skin or indirectly via the use of a cleansing puff, washcloth, sponge or other implement. The compositions are preferably diluted with water prior to, during, or after topical application, and then subsequently the skin or hair rinsed or wiped off, preferably rinsed off of the applied surface using water or a water-insoluble substrate in combination with water.

The present invention is therefore also directed to methods of cleansing the skin through the above-described application of the compositions of the present invention. The methods of the present invention are also directed to a method of providing effective delivery of the desired skin active agent, and the resulting benefits from such effective delivery as described herein, to the applied surface through the above-described application of the compositions of the present invention. Preferably, the method is directed to delivering skin benefits to skin or hair, said method comprising the steps of: dispensing an effective amount of a multi-phase personal care composition according to the multi-phased personal care composition of the present invention onto an implement selected from the group consisting of a cleansing puff, washcloth, sponge, and human hand; topically applying said composition to said skin or hair using said implement; and removing said composition from said skin or hair by rinsing said skin or hair

Method Of Manufacture

The multi-phase personal care compositions may be prepared by any known or otherwise effective technique, suitable for making and formulating the desired multi-phase product form. It is effective to combine toothpaste-tube filling technology with a spinning stage design. Additionally, the present invention can be prepared by the method and apparatus as disclosed in U.S. Pat. No. 6,213,166. The method and apparatus allows two or more compositions to be filled with a spiral configuration into a single container. The method requires that at least two nozzles be employed to fill the container. The container is placed on a static mixer and spun as the composition is introduced into the container.

Alternatively, it is effective to combine at least two phases by first placing the separate compositions in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. Specific non-limiting examples of such methods as they are applied to specific embodiments of the present invention are described in the following examples.

If the multi-phase personal care compositions comprises patterns of varying colors it can be desirable to package these compositions in a transparent or translucent package such that the consumer can view the pattern through the package. Because of the viscosity of the subject compositions it may also be desirable to include instructions to the consumer to store the package upside down, on its cap to facilitate dispensing.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

Each of the examples below are of multi-phase personal care compositions comprising 50%, by weight of the personal care composition, of a first phase and 50%, by weight of the multi-phase personal care composition, of a second phase. The amount of each component in a particular phase is provided as a weight percent based on the weight of the particular phase that contains the component.

The following comparative examples demonstrate how much of the opaque structured thermodynamic domain has been destroyed when a colorant has Barium and/or Aluminum, are present in the multi-phased personal care composition versus the multi-phased personal care composition comprising a colorant that is substantially free of Barium and/or Aluminum.

### Examples

The following examples described in Table 1 are non-limiting examples of multi-phased personal care compositions of the present invention.

TABLE 1
Cleansing Phase and Benefit phase Compositions

| | Example 1 | Example 2 | Comparative Example A | Comparative Example B |
|---|---|---|---|---|
| Ingredient | wt % | wt % | wt % | wt % |
| I. Cleansing Phase | | | | |
| Miracare SLB-365 (from Rhodia) (Sodium Trideceth | 47.4 | 47.4 | 47.4 | 47.4 |
| Sulfate, Sodium Lauramphoacetate, Cocamide MEA) | | | | |
| Guar Hydroxypropyltrimonium Chloride | 0.7 | 0.7 | 0.7 | 0.7 |
| (N-Hance 3196 from Aqualon) | | | | |
| PEG 90M (Polyox WSR 301 from Dow Chemical) | 0.1 | 0.1 | 0.1 | 0.1 |
| Glycerin | 0.3 | 0.3 | 0.3 | 0.3 |
| Sodium Chloride | 3.5 | 3.5 | 3.5 | 3.5 |
| Disodium EDTA | 0.05 | 0.05 | 0.05 | 0.05 |
| Glydant | 0.67 | 0.67 | 0.67 | 0.67 |
| Citric Acid | 0.4 | 0.4 | 0.4 | 0.4 |
| Perfume | 2.0 | 2.0 | 2.0 | 2.0 |
| Expancel 091 DE 40 d30 (from Expancel, Inc.) | 0.45 | 0.45 | 0.45 | 0.45 |
| Water | Q.S. | Q.S. | Q.S. | Q.S. |
| (pH) | (6.0) | (6.0) | (6.0) | (6.0) |
| II. Benefit phase | | | | |
| Petrolatum (Superwhite Protopet from WITCO) | 60 | 60 | 60 | 60 |
| Mineral Oil (Hydrobrite 1000 PO White MO from WITCO) | 39.90 | 39.90 | 39.90 | 39.90 |
| Red 7 Ca Lake | 0.10 | — | — | — |
| Red 34 Ca Lake | | 0.10 | | |
| Red 6 Barium Lake | — | — | 0.10 | — |
| Red 40 Al Lake | — | — | — | 0.10 |
| Stability (5 days @ 120 F.) | Pass | Pass | Fail | Fail |

The multi-phase personal care compositions described above can be prepared by conventional formulation and mixing techniques. Prepare the cleansing phase composition by first add citric acid into water at 1:3 ratio to form a citric acid premix. Disperse Polyox WSR 301 in glycerin at 1:3 ratio to form a Polyox premix. Disperse Polymer N-Hance in water at 1:10 ratio to form a N-Hance premix. Then, add the following ingredients into the main mixing vessel in the following sequence: water, N-Hance premix, Polyox premix, citric acid premix, and Expancel. Start agitation of the mixing vessel. Then, add Miracare SLB-365, sodium chloride, sodium benzoate, Disodium EDTA, glydant, and perfume. Keep agitation until a homogenous solution forms.

Prepare the benefit phase by first, disperse colorant in Hydrobrite 1000 White Mineral at 1:10 ratio. Mix well the premix using a high shear mixer. Add, SuperWhite Protopet petrolatum into a mixing vessel. Heat the vessel to 190° F. Then, add Hydrobrite 1000 White Mineral Oil and colorant premix into the mixing vessel with agitation. Let the vessel cool down with slow agitation. The cleansing and benefit phases are co-mixed at 50:50 w/w ratio and stored at 120 F. for 5 days.

Then, the composition is ultracentrifuged @50,000 rpm for 18 hours at 25° C. The Structured Domain Volume Ratio is measured after ultracentrifugation. Example 1 and 2 pass phase stability test with <10% structured phase degradation after 5 days @120 F. The comparative examples A and B failed phase stability test with >10% structured phase degradation.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A multi-phase personal care composition comprising: at least two visually distinct phases;
wherein at least one visually distinct phase comprises a cleansing phase; and
wherein at least one visually distinct phase comprises a colorant; and
wherein said colorant is substantially free of Barium and/or Aluminum; and
wherein said phases are packaged in physical contact with one another.

2. The multi-phase personal care composition of claim 1, comprising from about 0.00001% to about 10%, by weight the composition, of said colorant.

3. The multi-phase personal care composition of claim 1, wherein said colorants are selected from the group consisting of organic pigments, inorganic pigments, interference pigments, lakes, natural colorants, pearlescent agents, dyes, carmines, and mixtures thereof.

4. The multi-phase personal care composition of claim 1, wherein said colorant is UV stable.

5. The multi-phase personal care composition of claim 1, wherein said visually distinct phase are stable.

6. The multi-phase personal care composition of claim 1, wherein said cleansing phase further comprises a structured domain.

7. The multi-phase personal care composition of claim 6, wherein said structured domain is an opaque structured domain.

8. The multi-phase personal care composition of claim 7, wherein said opaque structured domain is a lamellar phase.

9. The multi-phase personal care composition of claim 1, wherein the cleansing phase comprises:
(i) at least one anionic surfactant;
(ii) at least one electrolyte;
(iii) at least one alkanolamide; and
(iv) water;
wherein the cleansing phase is non-Newtonian shear thinning; and
the cleansing phase has a viscosity of equal to or greater than about 3000 cps.

10. The multi-phase personal care composition of claim 1, further comprising a benefit phase.

11. The multi-phase personal care composition of claim 10, wherein said benefit phase comprises a hydrophobic composition; wherein said hydrophobic composition comprises from about 20% to about 100%, by weight of said benefit phase, of a hydrophobic component.

12. The multi-phase personal care composition of claim 1, wherein said visually distinct phases form a pattern.

13. The multi-phase personal care composition of claim 12, wherein said pattern is selected from the group consisting of striped, geometric, marbled and combinations thereof.

14. The multi-phase personal care composition of claim 1, wherein said composition additionally comprises skin care actives that are selected from the group consisting of vitamins and derivatives thereof; sunscreens; preservatives; anti-acne medicaments; antioxidants; skin soothing and healing; chelators and sequestrants; essential oils, skin sensates, and mixtures thereof.

15. The multi-phase personal care composition of claim 1, wherein at least one phase comprises a particle

16. A multi-phase personal care composition comprising: at least two visually distinct phases;
wherein at least one visually distinct phase comprises a cleansing phase comprising;
a) a surfactant component comprising;
i) at least one nonionic surfactant having an HLB from about 3.4 to about 15.0;
ii) at least one anionic surfactant;
iii) at least one amphoteric surfactant;
b) an electrolyte; and
wherein at least one visually distinct phase comprises a benefit phase; and
wherein at least one visually distinct phase comprises a colorant; and
wherein said colorant is substantially free of Barium and/or Aluminum; and
wherein said phases are packaged in physical contact with one another.

17. The multi-phase personal care composition of claim 16, comprising from about 0.00001% to about 10%, by weigh the composition, of said colorant.

18. The multi-phase personal care composition of claim 16, wherein said colorants are selected from the group consisting of organic pigments, inorganic pigments, interference pigments, lakes, natural colorants, pearlescent agents, dyes, carmines, and mixtures thereof.

19. The multi-phase personal care composition of claim 16, wherein said colorant is UV stable.

20. The multi-phase personal care composition of claim 16, wherein said visually distinct phase are stable.

21. The multi-phase personal care composition of claim 16, wherein said cleansing phase further comprises a structured domain.

22. The multi-phase personal care composition of claim 16, wherein said benefit phase comprises a hydrophobic composition; wherein said hydrophobic composition comprises from about 20% to about 100%, by weight of said benefit phase, of a hydrophobic component.

23. The multi-phase personal care composition of claim 16, wherein said visually distinct phases form a pattern.

24. The multi-phase personal care composition of claim 23, wherein said pattern is selected from the group consisting of striped, geometric, marbled and combinations thereof.

25. The multi-phase personal care composition of claim 16, wherein said composition additionally comprises skin care actives that are selected from the group consisting of vitamins and derivatives thereof; sunscreens; preservatives; anti-acne medicaments; antioxidants; skin soothing and healing; chelators and sequestrants; essential oils, skin sensates, and mixtures thereof.

26. The multi-phase personal care composition of claim 16, wherein at least one phase comprises a particle

27. A method of delivering skin benefits to skin or hair, said method comprising the steps of:
a) dispensing an effective amount of a multi-phase personal care composition according to claim 1 onto an implement selected from the group consisting of a cleansing puff, washcloth, sponge, and human hand;
b) topically applying said composition to said skin or hair using said implement; and
c) removing said composition from said skin or hair by rinsing said skin or hair with water.

28. A method of delivering skin benefits to skin or hair, said method comprising the steps of:
a) dispensing an effective amount of a multi-phase personal care composition according to claim 16 onto an implement selected from the group consisting of a cleansing puff, washcloth, sponge, and human hand;
b) topically applying said composition to said skin or hair using said implement; and
c) removing said composition from said skin or hair by rinsing said skin or hair with water.
